(19) United States
(12) Patent Application Publication
(10) Pub. No.: US 2025/0308096 A1
(43) Pub. Date: Oct. 2, 2025

(54) IMAGE PROCESSING APPARATUS, IMAGE PROCESSING METHOD, AND IMAGE PROCESSING PROGRAM

(71) Applicant: FUJIFILM CORPORATION, Tokyo (JP)

(72) Inventor: Takuya FUCHIGAMI, Tokyo (JP)

(21) Appl. No.: 19/082,188

(22) Filed: Mar. 18, 2025

(30) Foreign Application Priority Data
Mar. 26, 2024 (JP) 2024-049672

(51) Int. Cl. G06T 11/00; A61B 5/00; G06T 7/70; G06V 20/64

(52) U.S. Cl. CPC G06T 11/005; A61B 5/0037; A61B 5/743; G06T 7/70; G06V 20/64; G06T 2207/10088; G06T 2207/30012; G06T 2207/30084; G06T 2207/30172; G06T 2210/41; G06V 2201/031; G06V 2201/033

(57) ABSTRACT

A processor sets an imaging range based on a plurality of first tomographic images respectively representing a plurality of tomographic planes in a first direction for a subject and at least one second tomographic image representing a tomographic plane in a second direction intersecting the first direction. Accordingly, the imaging range can be accurately set.

### CROSS REFERENCE TO RELATED APPLICATIONS

The present application claims priority from Japanese Patent Application No. 2024-049672, filed on Mar. 26, 2024, the entire disclosure of which is incorporated herein by reference.

### BACKGROUND

### Technical Field

The present disclosure relates to an image processing apparatus, an image processing method, and an image processing program.

### Related Art

In recent years, advancements in medical devices such as a computed tomography (CT) apparatus and a magnetic resonance imaging (MRI) apparatus have enabled the use of a higher-quality and high-resolution image with a small slice thickness for image diagnosis.

In a case in which a subject is imaged using an imaging apparatus such as a CT apparatus or an MRI apparatus, scout imaging is performed prior to main imaging for acquiring an image with a small slice thickness (or a small slice interval) in order to determine an imaging range, and a positioning image (scout image) with a relatively larger slice thickness (or a larger slice interval) than that of the image of the main imaging is acquired. An operator of the imaging apparatus, such as a technician, sets an imaging range for the main imaging while viewing the scout image.

Meanwhile, setting the imaging range while viewing the scout image requires the operator to perform the setting manually, which may be time-consuming. In addition, the accuracy of the setting may vary because the accuracy of the setting depends on the operator's skill and experience. Therefore, various methods have been proposed to automatically set the imaging range from the scout image. For example, a method has been proposed in which a landmark is detected from each of scout images of an axial plane, a sagittal plane, and a coronal plane, and an imaging range for each of the axial plane, the sagittal plane, and the coronal plane is determined using the landmark (refer to the Internet <URL: https://www.siemens-healthineers.com/en-us/magnetic-resonance-imaging/technologies-and-i nnovations/dotgo-workflow>). Additionally, a method has been proposed in which, for an MRI image of the spine, intervertebral disc regions are extracted from each of a plurality of sagittal images, a three-dimensional intervertebral disc region is extracted based on the plurality of extracted two-dimensional intervertebral disc regions, and an imaging range of an intervertebral disc image is set based on the extracted three-dimensional intervertebral disc region (refer to JP2014-121598A).

Since the method described in JP2014-121598A sets the imaging range using only the sagittal image, the imaging range in planes other than the sagittal plane cannot be accurately set.

### SUMMARY OF THE INVENTION

The present disclosure has been made in view of the above-described circumstances, and an object of the present disclosure is to enable accurate setting of an imaging range.

According to the present disclosure, there is provided an image processing apparatus comprising a processor.

The processor is configured to set an imaging range based on a plurality of first tomographic images respectively representing a plurality of tomographic planes in a first direction for a subject and at least one second tomographic image representing a tomographic plane in a second direction intersecting the first direction.

According to the present disclosure, there is provided an image processing method comprising: causing a computer to execute: setting an imaging range based on a plurality of first tomographic images respectively representing a plurality of tomographic planes in a first direction for a subject and at least one second tomographic image representing a tomographic plane in a second direction intersecting the first direction.

According to the present disclosure, there is provided an image processing program for causing a computer to execute a procedure comprising: setting an imaging range based on a plurality of first tomographic images respectively representing a plurality of tomographic planes in a first direction for a subject and at least one second tomographic image representing a tomographic plane in a second direction intersecting the first direction.

According to the present disclosure, the imaging range can be accurately set.

### BRIEF DESCRIPTION OF THE DRAWINGS

FIG. 1 is a perspective view showing an outline of an MRI apparatus to which an image processing apparatus according to a first embodiment of the present disclosure is applied.

FIG. 2 is a diagram showing a hardware configuration of the image processing apparatus according to the first embodiment.

FIG. 3 is a diagram showing a functional configuration of the image processing apparatus according to the first embodiment.

FIG. 4 is a diagram illustrating derivation of a landmark in a first tomographic image.

FIG. 5 is a diagram illustrating derivation of a landmark in a second tomographic image.

FIG. 6 is a diagram illustrating derivation of presence information related to a structure included in a sagittal image.

FIG. 7 is a diagram showing a plane passing through an intervertebral disc located between adjacent vertebrae included in the sagittal image.

FIG. 8 is a diagram illustrating the derivation of the presence information related to the structure included in the sagittal image.

FIG. 9 is a diagram illustrating another derivation of the presence information related to the structure included in the sagittal image.

FIG. 10 is a diagram illustrating a positional relationship between the sagittal image and a coronal image.

FIG. 11 is a diagram illustrating derivation of presence information related to a structure included in the coronal image.

FIG. 12 is a diagram showing a straight line passing through an intervertebral disc included in the coronal image.

FIG. 13 is a diagram illustrating the derivation of the presence information related to the structure included in the coronal image.

FIG. 14 is a diagram illustrating another derivation of the presence information related to the structure included in the coronal image.

FIG. 15 is a diagram illustrating setting of a spinal centerline in the sagittal image.

FIG. 16 is a diagram illustrating setting of an imaging range for the structure included in the coronal image.

FIG. 17 is a diagram showing a displayed scout image.

FIG. 18 is a diagram showing another displayed scout image.

FIG. 19 is a flowchart showing processing performed in the first embodiment.

FIG. 20 is a diagram showing a functional configuration of an image processing apparatus according to a second embodiment.

FIG. 21 is a diagram illustrating learning of a derivation model.

FIG. 22 is a diagram illustrating processing performed by a first derivation unit in the second embodiment.

FIG. 23 is a flowchart showing processing performed in the second embodiment.

FIG. 24 is a diagram illustrating setting of an imaging range using a scout image during MRI imaging of kidneys.

FIG. 25 is a diagram showing a scout image in a case in which a size of one kidney in a direction perpendicular to an axial plane is small.

FIG. 26 is a diagram illustrating derivation of a kidney region in a coronal image in a third embodiment.

FIG. 27 is a diagram illustrating derivation of a kidney region in an axial image in the third embodiment.

FIG. 28 is a diagram illustrating derivation of presence information related to the kidneys included in the coronal image.

FIG. 29 is a diagram illustrating derivation of presence information related to the kidney included in the axial image.

FIG. 30 is a diagram illustrating setting of the imaging range in the third embodiment.

FIG. 31 is a flowchart showing processing performed in the third embodiment.

### DETAILED DESCRIPTION

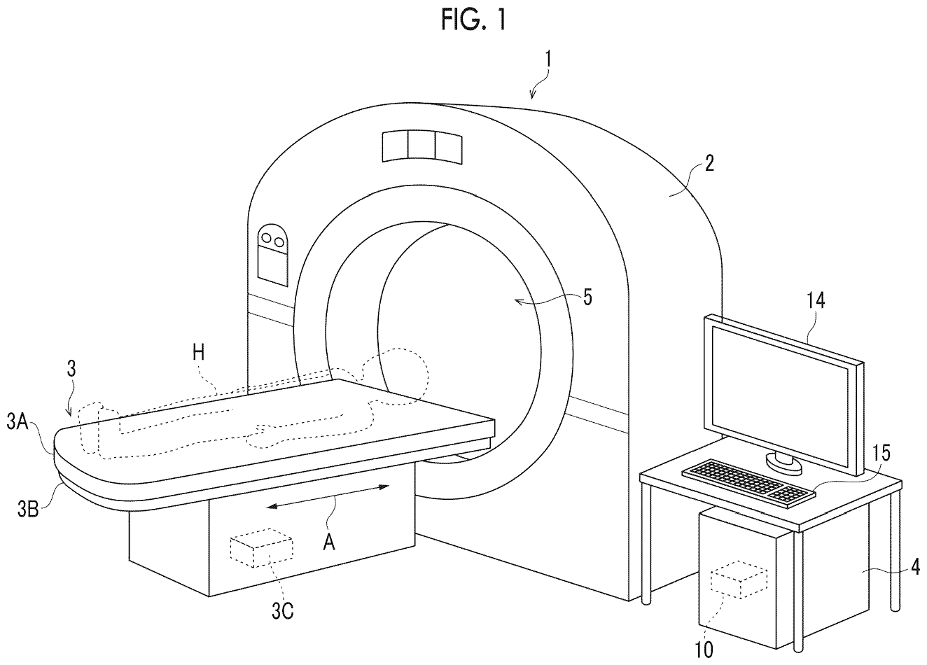

Hereinafter, embodiments of the present disclosure will be described with reference to the drawings. FIG. 1 is a perspective view showing an outline of an imaging apparatus to which an image processing apparatus according to a first embodiment of the present disclosure is applied. As shown in FIG. 1, the imaging apparatus according to the present embodiment is an MRI apparatus 1, and comprises a gantry 2, a patient table 3, and a console 4.

The gantry 2 has a tunnel-shaped structure including an opening portion 5 at a center thereof. A magnetic field generation unit including a static magnetic field magnet, a radiofrequency magnetic field coil, and a gradient magnetic field coil is incorporated in the gantry 2 (all not shown). A receive coil (not shown) is disposed in the patient table 3. The receive coil receives a nuclear magnetic resonance signal emitted from an imaging site of a subject H due to a radiofrequency magnetic field. A nuclear magnetic resonance image, that is, an MRI image, is generated based on the nuclear magnetic resonance signal received by the receive coil.

The patient table 3 includes a patient table portion 3A on which the subject lies, a base portion 3B that supports the patient table portion 3A, and a drive unit 3C that reciprocates the patient table portion 3A in a direction of an arrow A. The patient table portion 3A is slidable relative to the base portion 3B in the direction of the arrow A by the drive unit 3C. Upon capturing the MRI image, the patient table portion 3A slides, and the subject H lying on the patient table portion 3A is transported into the opening portion 5 of the gantry 2.

Imaging of the subject by the drive of the gantry 2 and the drive of the patient table 3 is performed in response to an input from an operator via the console 4. The console 4 incorporates an image processing apparatus according to the first embodiment.

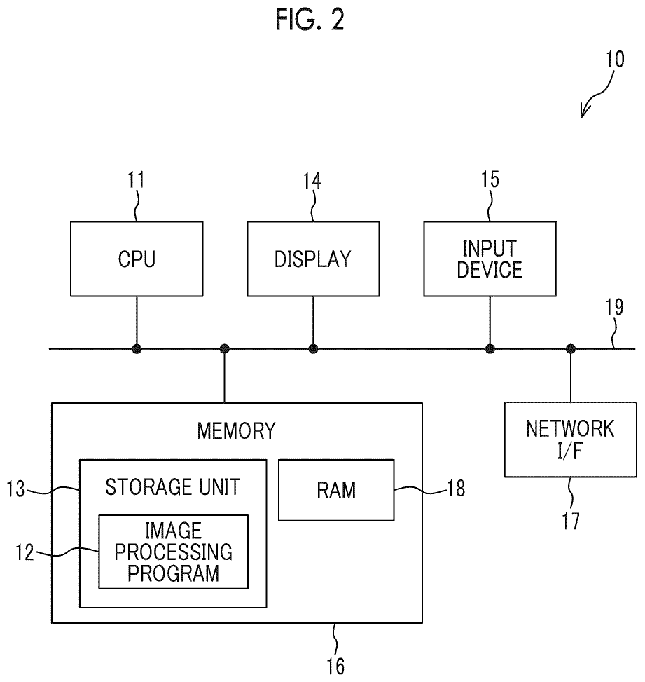

Next, the image processing apparatus according to the first embodiment incorporated in the console 4 will be described. First, a hardware configuration of the image processing apparatus according to the first embodiment will be described with reference to FIG. 2. As shown in FIG. 2, an image processing apparatus 10 includes a central processing unit (CPU) 11, a display 14, an input device 15, a memory 16, and a network interface (I/F) 17 connected to a network (not shown). The CPU 11, the display 14, the input device 15, the memory 16, and the network I/F 17 are connected to a bus 19. The CPU 11 is an example of a processor in the present disclosure.

The memory 16 includes a storage unit 13 and a random access memory (RAM) 18. The RAM 18 is a memory for primary storage and is, for example, a RAM such as a static random access memory (SRAM) or a dynamic random access memory (DRAM).

The storage unit 13 is a non-volatile memory and is implemented by, for example, at least one of a hard disk drive (HDD), a solid state drive (SSD), an electrically erasable and programmable read only memory (EEPROM), a flash memory, or the like. An image processing program 12 according to the present embodiment is stored in the storage unit 13 as a storage medium. The CPU 11 reads the image processing program 12 from the storage unit 13, loads the read image processing program 12 into the RAM 18, and executes the loaded image processing program 12.

The display 14 is a device that displays various screens and is, for example, a liquid crystal display or an electro luminescence (EL) display. The input device 15 is a device for user input and is, for example, at least any of a keyboard, a mouse, a microphone for audio input, a touchpad for proximity input including a contact, or a camera for gesture input. The network I/F 17 is an interface for connecting to the network.

The image processing program 12 is stored in a storage device of a server computer connected to the network or in a network storage in an externally accessible state and is downloaded and installed on a computer that constitutes the image processing apparatus 10 in response to a request. Alternatively, the image processing program 12 is distributed by being recorded on a recording medium such as a digital versatile disc (DVD) or a compact disc read only memory (CD-ROM) and is then installed onto the computer that constitutes the image processing apparatus 10 from the recording medium.

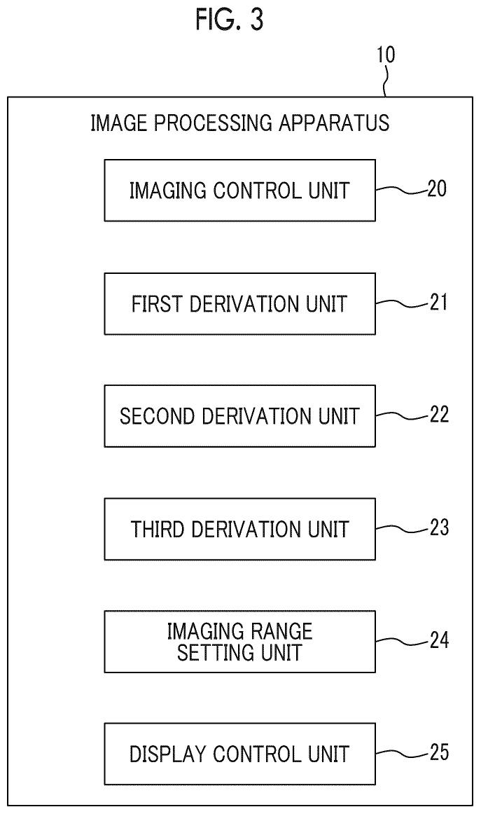

Next, a functional configuration of the image processing apparatus according to the first embodiment will be described. FIG. 3 is a diagram showing the functional configuration of the image processing apparatus according to the first embodiment. As shown in FIG. 3, the image processing apparatus 10 comprises an imaging control unit 20, a first derivation unit 21, a second derivation unit 22, a third derivation unit 23, an imaging range setting unit 24, and a display control unit 25. The CPU 11 executes the image processing program 12, whereby the CPU 11 functions as the imaging control unit 20, the first derivation unit 21, the second derivation unit 22, the third derivation unit 23, the imaging range setting unit 24, and the display control unit 25.

The imaging control unit 20 controls the magnetic field generation unit provided in the gantry 2 and the receive coil provided in the patient table 3 to perform imaging of the subject H in response to an instruction via the input device 15. Upon MRI imaging, scout imaging is performed prior to main imaging for acquiring an MRI image with a small slice thickness in order to determine an imaging range. The scout imaging is performed by imaging the subject H to acquire several (for example, three) tomographic images along a predetermined imaging direction of the subject H. The tomographic image is, for example, an image showing at least one of an axial plane, a sagittal plane, or a coronal plane. The imaging direction refers to a direction perpendicular to each of the axial plane, the sagittal plane, and the coronal plane.

In the first embodiment, it is assumed that the MRI image of the spine of the subject H is acquired, and imaging ranges for a plurality of vertebrae and a plurality of intervertebral discs included in the spine are set. In order to set the imaging range, in the first embodiment, scout imaging for setting the imaging range is performed before the main imaging, and a sagittal image and a coronal image, which are tomographic images of the sagittal plane and the coronal plane of the subject H, are acquired as scout images.

The scout image acquired through the scout imaging includes a plurality of tomographic images with a relatively larger slice thickness than that of the MRI image acquired through the main imaging. In the present embodiment, the imaging range for the main imaging is automatically set based on the scout image. The setting of the imaging range will be described below. After the imaging range is set, the operator inputs an instruction for the main imaging via the input device 15, and the imaging control unit 20 performs the main imaging. Consequently, the MRI image of the subject H is acquired. The MRI image acquired through the main imaging includes a plurality of tomographic images with a relatively smaller slice thickness than that of the scout image. For example, the slice thickness of the scout image is 3 mm or greater, and the slice thickness of the MRI image of the main imaging is approximately 1 mm. The scout image acquired through the scout imaging and the MRI image acquired through the main imaging are stored in the storage unit 13.

The first derivation unit 21 derives a landmark of a structure included in a plurality of first tomographic images respectively representing a plurality of tomographic planes in a first direction for the subject H. In the first embodiment, the first direction is a direction perpendicular to the sagittal plane, the plurality of tomographic planes in the first direction are sagittal planes, and the first tomographic image is a sagittal image. Additionally, the first derivation unit 21 derives a landmark of a vertebra included in the sagittal image. The landmark derived in the sagittal image is an example of a first position. As the landmark of the vertebra, four corner points of a vertebral body that constitutes the vertebra are used. For example, the first derivation unit 21 derives the landmark of the vertebra using a detection model that has undergone machine learning to detect the four corner points of the vertebral body from the sagittal image.

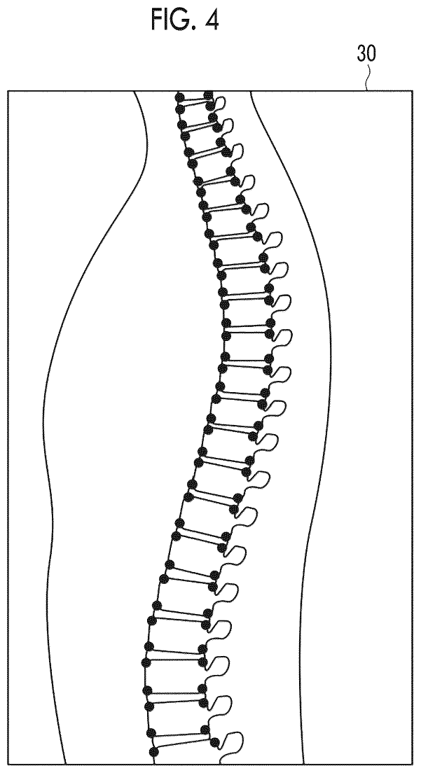

FIG. 4 is a diagram illustrating the derivation of the landmark in the first tomographic image. As shown in FIG. 4, the first derivation unit 21 uses the detection model to derive the four corner points of the vertebral body that constitutes the vertebra as the landmarks of the vertebra in a sagittal image 30 including the spine. In FIG. 4, the derived landmarks are indicated by black circles.

The second derivation unit 22 derives a landmark of a structure included in a plurality of second tomographic images respectively representing a plurality of tomographic planes in a second direction for the subject H. In the first embodiment, the second direction is a direction perpendicular to the coronal plane, the plurality of tomographic planes in the second direction are coronal planes, and the second tomographic image is a coronal image. In addition, the second derivation unit 22 derives a landmark of the vertebra included in the coronal image. The landmark derived in the coronal image is an example of a second position. As the landmark of the vertebra, four corner points of a vertebral body that constitutes the vertebra are used, similar to the first derivation unit 21. For example, the second derivation unit 22 derives the landmark of the vertebra using a detection model that has undergone machine learning to detect the four corner points of the vertebral body from the coronal image.

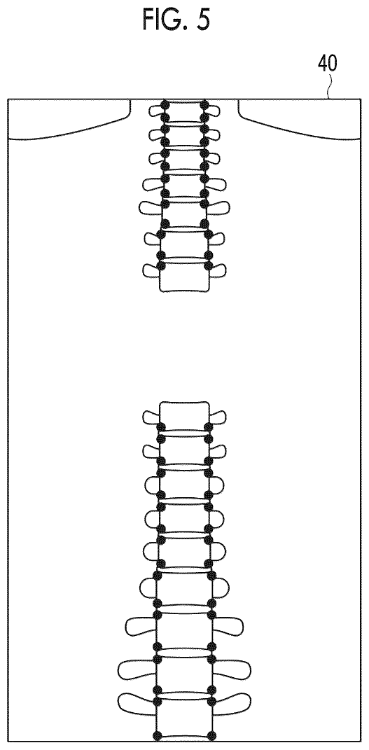

FIG. 5 is a diagram illustrating the derivation of the landmark in the second tomographic image. As shown in FIG. 5, the second derivation unit 22 uses the detection model to derive the four corner points of the vertebral body that constitutes the vertebra as the landmarks of the vertebra in a coronal image 40 including the spine. In FIG. 5, the derived landmarks are also indicated by black circles.

The third derivation unit 23 derives presence information related to the structure in the tomographic plane in the second direction based on the landmarks derived by the first derivation unit 21 and the landmarks derived by the second derivation unit 22. Here, an intervertebral disc is present between the vertebrae. In the first embodiment, the third derivation unit 23 derives an orientation and a presence range of at least one of the vertebra or the intervertebral disc as the presence information related to the structure, based on the landmarks of the vertebra included in the sagittal image and the landmarks of the vertebra included in the coronal image. First, the derivation of the presence information related to the structure included in the sagittal image will be described. In the following description, it is assumed that the presence information related to the intervertebral disc is derived as the presence information related to the structure.

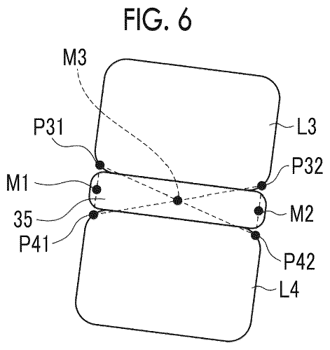

FIG. 6 is a diagram illustrating the derivation of the presence information related to the structure included in the sagittal image. FIG. 6 shows a third lumbar vertebra L3, a fourth lumbar vertebra L4, and an intervertebral disc 35 located between the third lumbar vertebra L3 and the fourth lumbar vertebra L4. First, the third derivation unit 23 derives a midpoint of the landmarks of two opposing vertebrae by using the landmarks derived in the sagittal image. For example, the third derivation unit 23 derives a midpoint M1 between a landmark P31 at a lower left corner of the third lumbar vertebra L3 and a landmark P41 at an upper left corner of the fourth lumbar vertebra L4, and a midpoint M2 between a landmark P32 at a lower right corner of the third lumbar vertebra L3 and a landmark P42 at an upper right corner of the fourth lumbar vertebra L4, which are included in the sagittal image. In addition, the third derivation unit 23 derives a centroid M3 of the landmark P31 at the lower left corner of the third lumbar vertebra L3, the landmark P32 at the lower right corner of the third lumbar vertebra L3, the landmark P41 at the upper left corner of the fourth lumbar vertebra L4, and the landmark P42 at the upper right corner of the fourth lumbar vertebra L4.

Since a plurality of sagittal images 30 are acquired, the third derivation unit 23 performs the same processing as that in FIG. 6 on the third lumbar vertebra L3 and the fourth lumbar vertebra L4 included in each of the plurality of sagittal images 30, thereby deriving the midpoints M1 and M2 and the centroid M3 of the landmarks of the third lumbar vertebra L3 and the fourth lumbar vertebra L4 in each of the plurality of sagittal images 30. The third derivation unit 23 derives a plane passing between the third lumbar vertebra L3 and the fourth lumbar vertebra L4, that is, a plane passing through the intervertebral disc 35, by performing plane fitting on the midpoints M1 and M2 and the centroid M3 of the landmarks of the third lumbar vertebra L3 and the fourth lumbar vertebra L4 derived for the plurality of sagittal images 30.

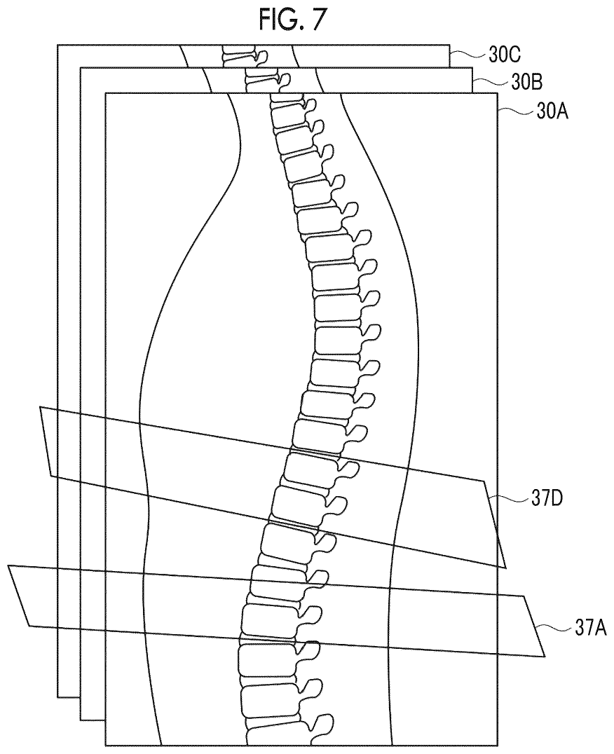

The third derivation unit 23 derives a plane passing through the intervertebral disc located between adjacent vertebrae in the plurality of sagittal images 30 by performing the same processing as described above on the plurality of vertebrae included in the plurality of sagittal images 30. FIG. 7 is a diagram showing the plane passing through the intervertebral disc located between adjacent vertebrae. In FIG. 7, planes derived for three sagittal images 30A to 30C are shown. The third derivation unit 23 derives planes for all the intervertebral discs located between adjacent vertebrae; however, in FIG. 7, all the planes are not shown, and only a plane 37A passing through the intervertebral disc between the fourth lumbar vertebra L4 and the third lumbar vertebra L3 and a plane 37D passing through the intervertebral disc between a first lumbar vertebra L1 and a twelfth thoracic vertebra T12 are shown.

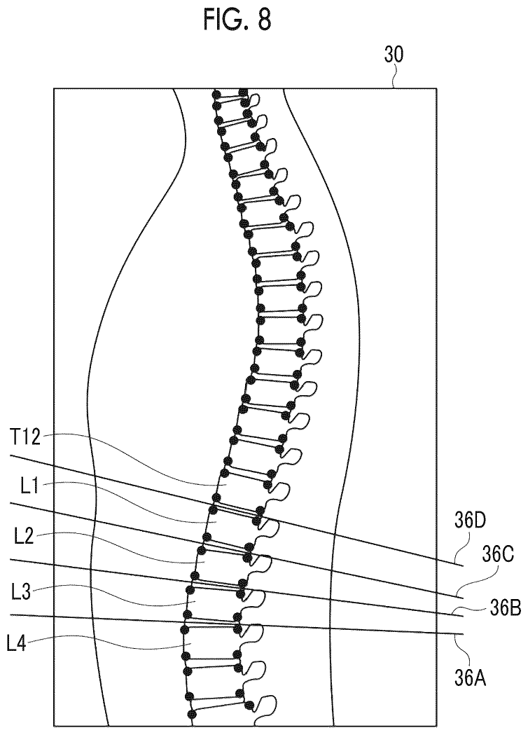

Meanwhile, in each of the sagittal images 30A to 30C, the plane passing through the intervertebral disc is indicated by a straight line as shown in FIG. 8. In FIG. 8, straight lines passing through all the intervertebral discs are not shown, and only a straight line 36A passing through the intervertebral disc between the fourth lumbar vertebra L4 and the third lumbar vertebra L3, a straight line 36B passing through the intervertebral disc between the third lumbar vertebra L3 and a second lumbar vertebra L2, a straight line 36C passing through the intervertebral disc between the second lumbar vertebra L2 and the first lumbar vertebra L1, and a straight line 36D passing through the intervertebral disc between the first lumbar vertebra L1 and the twelfth thoracic vertebra T12 are shown.

As mentioned above, the planes 37A and 37D (the straight lines 36A to 36D in the sagittal image) passing through the intervertebral discs derived by the third derivation unit 23 are examples of the presence information representing the orientation and the presence range of the intervertebral disc, that is, the presence information related to the structure.

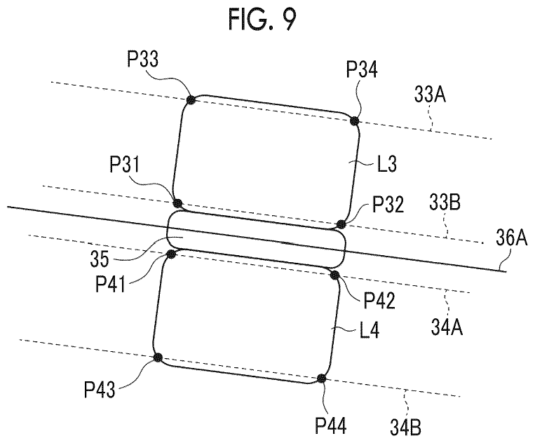

In the present embodiment, the third derivation unit 23 derives the presence information from the midpoints and the centroid of the landmarks for the adjacent vertebrae for the sagittal image 30, but the present disclosure is not limited thereto. FIG. 9 is a diagram illustrating another derivation of the presence information. As shown in FIG. 9, the third derivation unit 23 derives a straight line 33A passing through two landmarks P33 and P34 on an upper side of the third lumbar vertebra L3 and a straight line 33B passing through two landmarks P31 and P32 on a lower side of the third lumbar vertebra L3. Additionally, for the fourth lumbar vertebra L4, the third derivation unit 23 derives a straight line 34A passing through two landmarks P41 and P42 on an upper side of the fourth lumbar vertebra L4 and a straight line 34B passing through two landmarks P43 and P44 on a lower side of the fourth lumbar vertebra LA. Further, the third derivation unit 23 derives the straight line 36A that bisects the straight line 33B derived on the lower side of the third lumbar vertebra L3 and the straight line 34A derived on the upper side of the fourth lumbar vertebra LA. The straight line 36A, which passes through the intervertebral disc, is a straight line indicating the orientation and the presence range of the intervertebral disc.

The third derivation unit 23 performs the same processing as that in FIG. 9 for all the vertebrae included in the sagittal image 30, thereby deriving the straight lines passing through two landmarks on the upper side and the lower side of all the vertebrae in the sagittal image 30 and deriving the straight line that bisects two opposing straight lines of adjacent vertebrae, that is, the straight line passing through the intervertebral disc. As a result, as shown in FIG. 8, for all the sagittal images, straight lines (only the straight lines 36A to 36D are shown in FIG. 8) passing through the intervertebral discs are derived as the presence information related to the intervertebral discs.

Here, the plurality of sagittal images 30 are acquired. Therefore, the third derivation unit 23 derives, for all the sagittal images, the straight lines passing through two landmarks on the upper side and the lower side of all the vertebrae and the straight line passing through the intervertebral disc based on the two straight lines. In this case, the third derivation unit 23 can derive the plane passing through the intervertebral disc (only the planes 37A and 37D are shown in FIG. 7) as shown in FIG. 7 by performing plane fitting on the straight lines 36A to 36D derived for the same intervertebral disc in the three sagittal images 30A to 30C.

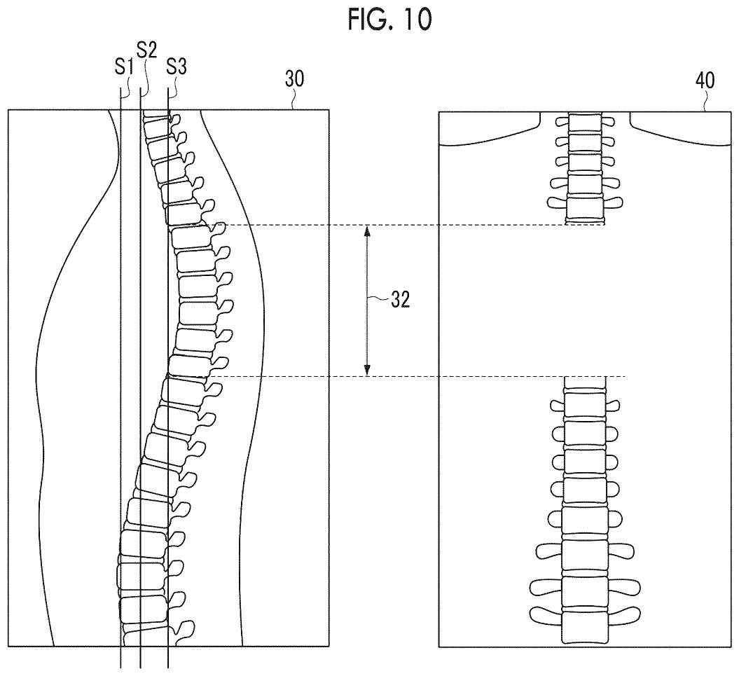

Here, a positional relationship between the sagittal image and the coronal image acquired as the scout images will be described. FIG. 10 is a diagram illustrating the positional relationship between the sagittal image and the coronal image. Several (for example, three) scout images are acquired for each tomographic plane at relatively large slice intervals. As shown in FIG. 10, it is assumed that the coronal images are acquired for coronal planes S1, S2, and S3 indicated by straight lines in the sagittal image 30. The human spinal column is curved in an anterior-posterior direction of the human body. Therefore, all the vertebrae are not included in the coronal image in any of the coronal planes S1 to S3. In FIG. 10, the coronal image 40 of the coronal plane S3 is shown. In the coronal image 40 shown in FIG. 10, the vertebrae included in a range 32 in the sagittal image 30 are not included.

For the vertebrae included in the coronal image 40, the third derivation unit 23 derives the presence information related to the structure in the tomographic plane in the second direction based on the landmarks derived by the second derivation unit 22.

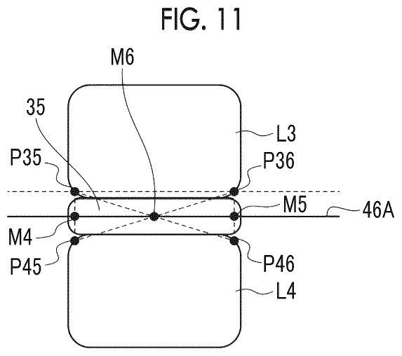

FIG. 11 is a diagram illustrating the derivation of the presence information related to the structure included in the coronal image. FIG. 11 shows the third lumbar vertebra L3, the fourth lumbar vertebra L4, and the intervertebral disc 35 located between the third lumbar vertebra L3 and the fourth lumbar vertebra L4, similar to FIG. 6. First, the third derivation unit 23 derives a midpoint of the landmarks of two opposing vertebrae by using the landmarks derived in the coronal image. For example, the third derivation unit 23 derives a midpoint M4 between a landmark P35 at the lower left corner of the third lumbar vertebra L3 and a landmark P45 at the upper left corner of the fourth lumbar vertebra L4, and a midpoint M5 between a landmark P36 at the lower right corner of the third lumbar vertebra L3 and a landmark P46 at the upper right corner of the fourth lumbar vertebra L4, which are included in the coronal image. In addition, the third derivation unit 23 derives a centroid M6 of the landmark P35 at the lower left corner of the third lumbar vertebra L3, the landmark P36 at the lower right corner of the third lumbar vertebra L3, the landmark P45 at the upper left corner of the fourth lumbar vertebra LA, and the landmark P46 at the upper right corner of the fourth lumbar vertebra L4. The third derivation unit 23 derives a straight line 46A passing through the midpoint M4 and the midpoint M5. The straight line 46A is a straight line passing through the intervertebral disc 35. The centroid M6 will be described below.

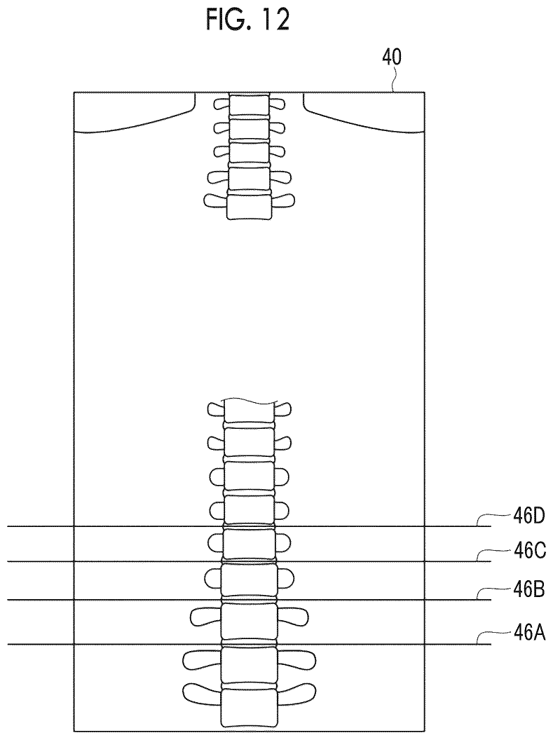

The third derivation unit 23 performs the same processing as that in FIG. 11 for all the vertebrae included in the coronal image 40, thereby deriving straight lines passing through the intervertebral discs between all the vertebrae in the coronal image 40. FIG. 12 is a diagram showing the straight line passing through the intervertebral disc included in the coronal image. In FIG. 12, straight lines passing through all the intervertebral discs are not shown, and only the straight line 46A passing through the intervertebral disc between the fourth lumbar vertebra L4 and the third lumbar vertebra L3, a straight line 46B passing through the intervertebral disc between the third lumbar vertebra L3 and the second lumbar vertebra L2, a straight line 46C passing through the intervertebral disc between the second lumbar vertebra L2 and the first lumbar vertebra L1, and a straight line 46D passing through the intervertebral disc between the first lumbar vertebra L1 and the twelfth thoracic vertebra T12 are shown. The straight line 46A is an example of the presence information representing the orientation and the presence range of the intervertebral disc, that is, the presence information related to the structure.

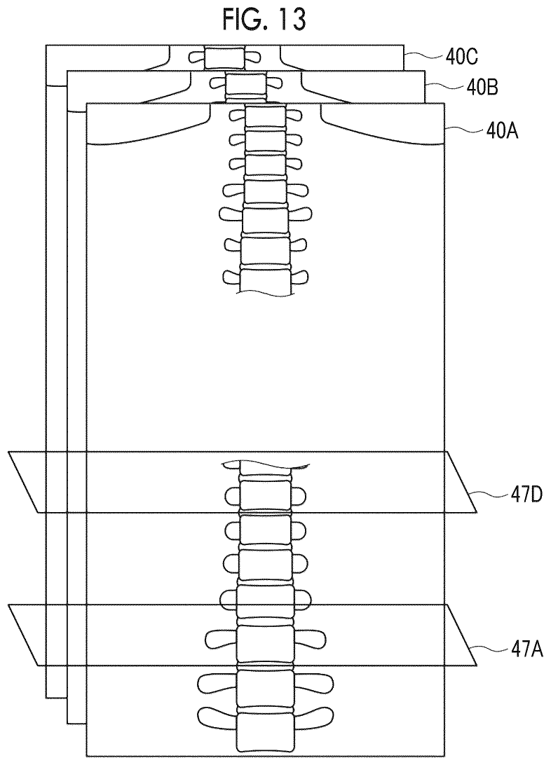

A plurality of (three in the present embodiment) coronal images are acquired through the scout imaging. Therefore, the third derivation unit 23 may derive straight lines passing through all the intervertebral discs for all the coronal images. In this case, as shown in FIG. 13, the third derivation unit 23 may derive the plane passing through the intervertebral disc by performing plane fitting on the straight lines passing through the same intervertebral disc derived from three coronal images 40A to 40C, and may derive the derived plane as the presence information representing the orientation and the presence range of the intervertebral disc, that is, the presence information related to the structure. The third derivation unit 23 derives planes for all the intervertebral discs located between adjacent vertebrae; however, in FIG. 13, all the planes are not shown, and only an imaging range 47A, which is a plane between the fourth lumbar vertebra L4 and the third lumbar vertebra L3, and an imaging range 47D, which is a plane between the first lumbar vertebra L1 and the twelfth thoracic vertebra T12, are shown. Upon the plane fitting, the centroid M6 derived as shown in FIG. 11 may be further used.

Meanwhile, as shown in FIG. 10, in the range 32 of the coronal image 40, the presence information related to the structure cannot be derived because the vertebra is not included. In the present embodiment, in the range 32 of the coronal image 40 shown in FIG. 10, the third derivation unit 23 derives an intersection line between the plane passing through the intervertebral disc derived in the sagittal image 30 and the coronal plane shown by the coronal image 40 as a straight line passing through the intervertebral disc for the coronal image 40.

In the present embodiment, as shown in FIG. 11, the midpoints M4 and M5 are derived from the landmarks of the vertebrae, and the straight line 46A passing through the midpoints M4 and M5 is derived as the straight line passing through the intervertebral disc 35, that is, the presence information related to the structure, but the present disclosure is not limited thereto. For example, correction may be performed by adding the midpoints M4 and M5 derived from the coronal image 40 to a point group used in deriving the plane passing through the intervertebral disc 35 in the sagittal image 30 and performing the plane fitting or the like, and the intersection line between the corrected plane and the coronal plane shown by the coronal image 40 may be derived as the straight line passing through the intervertebral disc 35.

Additionally, in the present embodiment, the centroid M3 and further the centroid M6 are derived as the presence range related to the vertebra from the landmarks of the vertebra, but the present disclosure is not limited thereto. For example, the centroid M3 acquired from the sagittal image 30 in which the likelihood of landmarks not being visible is relatively low may be corrected using the centroid M6 acquired from the coronal image 40 in which the likelihood of including a portion where landmarks are not visible is relatively high, and the corrected centroid M3 may be used as the presence range. Specifically, in a case in which the sagittal plane is represented by a Y axis and a Z axis and the coronal plane corresponds to an X axis and the Z axis, the X coordinate of the centroid M3 may be replaced with the X coordinate of the centroid M6.

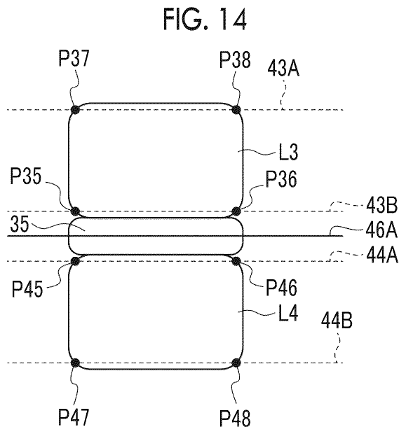

In the present embodiment, the third derivation unit 23 derives the presence information from the midpoint and the centroid of the landmarks for the adjacent vertebrae in relation to the coronal image 40, but the present disclosure is not limited thereto. FIG. 14 is a diagram illustrating another derivation of the presence information using the coronal image. As shown in FIG. 14, the third derivation unit 23 derives a straight line 43A passing through two landmarks P37 and P38 on the upper side of the third lumbar vertebra L3 and a straight line 43B passing through two landmarks P35 and P36 on the lower side of the third lumbar vertebra L3. In addition, for the fourth lumbar vertebra L4, the third derivation unit 23 derives a straight line 44A passing through two landmarks P45 and P46 on the upper side of the fourth lumbar vertebra L4 and a straight line 44B passing through two landmarks P47 and P48 on the lower side of the fourth lumbar vertebra L4. Further, the third derivation unit 23 derives the straight line 46A that bisects the straight line 43B derived on the lower side of the third lumbar vertebra L3 and the straight line 44A derived on the upper side of the fourth lumbar vertebra L4. The straight line 46A, which passes through the intervertebral disc, is a straight line indicating the orientation and the presence range of the intervertebral disc.

The third derivation unit 23 performs the same processing as that in FIG. 14 for all the vertebrae included in the coronal image 40, thereby deriving the straight lines passing through two landmarks on the upper side and the lower side of all the vertebrae in the coronal image 40 and deriving the straight line that bisects two opposing straight lines of adjacent vertebrae, that is, the straight line passing through the intervertebral disc. As a result, as shown in FIG. 12, straight lines (only the straight lines 46A to 46D are shown in FIG. 12) passing through the intervertebral discs included in the coronal image 40 are derived as the presence information related to the intervertebral discs.

Here, a plurality of coronal images 40 are acquired. Therefore, the third derivation unit 23 derives, for all the coronal images, the straight lines passing through two landmarks on the upper side and the lower side of all the vertebrae and the straight line passing through the intervertebral disc based on the straight lines. In this case, as shown in FIG. 13, the third derivation unit 23 can derive the plane (only the planes 47A and 47D are shown in FIG. 13) by performing plane fitting on the straight lines derived for the same intervertebral disc in the three coronal images 40A to 40C.

The imaging range setting unit 24 sets the imaging range for the next imaging, that is, the main imaging, based on the presence information related to the structure derived by the third derivation unit 23. Specifically, since the sagittal image and the coronal image are scout images, the imaging range for performing the main imaging is set to acquire the MRI image with a smaller slice thickness than that of the scout image.

Specifically, the imaging range setting unit 24 sets an inclination of the plane passing through the intervertebral disc with respect to the sagittal plane and an inclination of the straight line passing through the intervertebral disc with respect to the coronal plane as an inclination of the imaging plane and sets at least one of the centroid M3 or the centroid M6 as an imaging center. Here, the inclination of the plane passing through the intervertebral disc with respect to the sagittal plane refers to an inclination formed between an axial plane indicating a horizontal direction of the sagittal plane and the plane (for example, the planes 37A and 37D shown in FIG. 7). That is, the inclination is an inclination formed by an intersection line between the sagittal plane and the axial plane and an intersection line between the sagittal plane and the plane passing through the intervertebral disc. In addition, the inclination of the straight line (for example, the straight lines 46A to 46D shown in FIG. 12) passing through the intervertebral disc with respect to the coronal plane refers to an inclination formed by an intersection line between the axial plane indicating the horizontal direction of the coronal plane and the straight line passing through the intervertebral disc. Further, the imaging center refers to a position that is the center of an imaging region. Only the centroid M3 may be set as the imaging center.

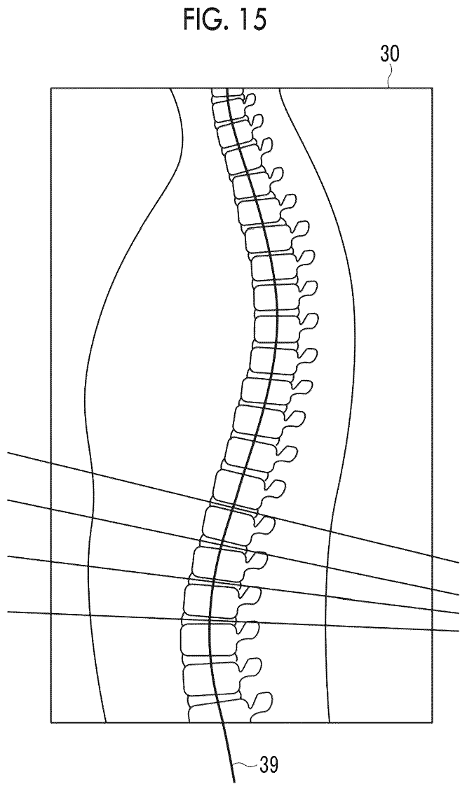

As shown in FIG. 15, the imaging range setting unit 24 may derive the centroids of the vertebral bodies from the landmarks of all the vertebral bodies in the sagittal image 30, derive, for example, a spinal centerline 39 to pass through the centroids by using a spline curve, and set the imaging range based on the spinal centerline 39. Specifically, in a case of the third lumbar vertebra L3 shown in FIG. 9, the centroid of the vertebral body refers to the centroid of four landmarks P31 to P34 of the third lumbar vertebra L3. Similarly, in the coronal image 40, the imaging range may also be set by deriving the spinal centerline 39. In this case, for the centroid of the vertebral body for which the landmark cannot be derived in the coronal image 40, the spinal centerline 39 in the coronal image 40 may be derived using the centroid of the vertebral body derived in the sagittal image 30. Additionally, specifically, the imaging range setting unit 24 may set a straight line or a plane orthogonal to the spinal centerline 39 as the imaging range.

In addition, the imaging range setting unit 24 may set the imaging range such that an angle formed by the imaging range with respect to a reference line or plane is within a predetermined range. For example, the imaging range may be set such that the straight line representing the imaging range on any of the sagittal image 30 or the coronal image 40, or the intersection line formed by any of the sagittal plane or the coronal plane and the plane indicating the imaging range, falls within a range of, for example, −20 degrees to +20 degrees with respect to the horizontal line of the sagittal image 30 or the coronal image 40, that is, the intersection line between any of the sagittal plane or the coronal plane and the axial plane.

Additionally, the imaging range setting unit 24 may set the imaging range such that an angle formed by the imaging ranges for adjacent intervertebral discs, that is, the imaging planes, falls within a predetermined range. For example, the imaging range may be set such that a difference in inclination between the straight lines representing adjacent imaging ranges, that is, the straight lines on any of the sagittal image 30 or the coronal image 40, or between the intersection lines between any of the sagittal plane or the coronal plane and the planes indicating the imaging ranges, falls within a range of −10 degrees to +10 degrees. In particular, in a case in which the angle formed by the imaging ranges for adjacent intervertebral discs, that is, the imaging planes, exceeds the predetermined range, the imaging range may be corrected such that the angle formed by the imaging ranges for the adjacent intervertebral discs falls within the predetermined range. As a result, it is possible to derive the presence range of the intervertebral disc such that the presence range derived for the adjacent intervertebral discs has a natural inclination.

In the above-described embodiment, the first derivation unit 21 and the second derivation unit 22 derive the four corner points of the vertebral body as the landmarks, but the present disclosure is not limited thereto. A point in an intervertebral disc region, for example, a point of any of a center position or a centroid position of the intervertebral disc may be derived as the landmark.

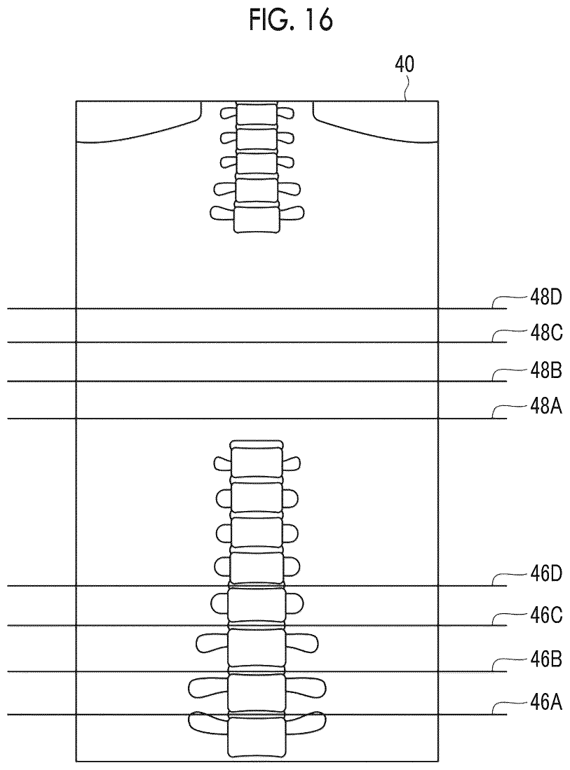

FIG. 16 is a diagram showing a coronal image in which the imaging range is set for a vertebra that is not included, that is, for an intervertebral disc. In FIG. 16, the imaging range is indicated by a straight line for the sake of description. As shown in FIG. 16, in addition to the imaging ranges 46A to 46D for the intervertebral discs between the vertebrae included in the coronal image 40, imaging ranges 48A to 48D for the intervertebral discs between the vertebrae that are not included in the coronal image 40 are shown in the coronal image 40. The imaging ranges 48A to 48D are derived by reflecting, in the coronal image 40, the presence information related to the intervertebral discs derived based on the sagittal image 30 as described above. In FIG. 16, only the straight lines representing the imaging ranges for some intervertebral discs are assigned, but in practice, straight lines representing the imaging ranges for all the intervertebral discs included in the coronal image 40 are assigned.

In the present embodiment, a case has been described in which the imaging range of the intervertebral disc is derived from the landmarks of the vertebra as the imaging range, but the imaging range of the vertebra may be derived. In this case, for example, in the sagittal image 30, a plane need only be derived from at least one of straight lines (for example, at least one of the straight line 33B or the straight line 34A in FIG. 9) connecting two landmarks located on an intervertebral disc side in adjacent vertebrae, and the inclination of the imaging plane in the sagittal plane need only be set based on the derived plane. Additionally, in the coronal image 40, a straight line (for example, at least one of the straight line 43B or the straight line 44A in FIG. 14) connecting two landmarks located on the intervertebral disc side in adjacent vertebrae need only be derived, and the inclination of the imaging plane in the coronal plane need only be set based on the derived straight line. Further, the centroid of four landmarks of the vertebra need only be set as the imaging center. The image for deriving the imaging center need only be at least one of the sagittal image 30 or the coronal image 40.

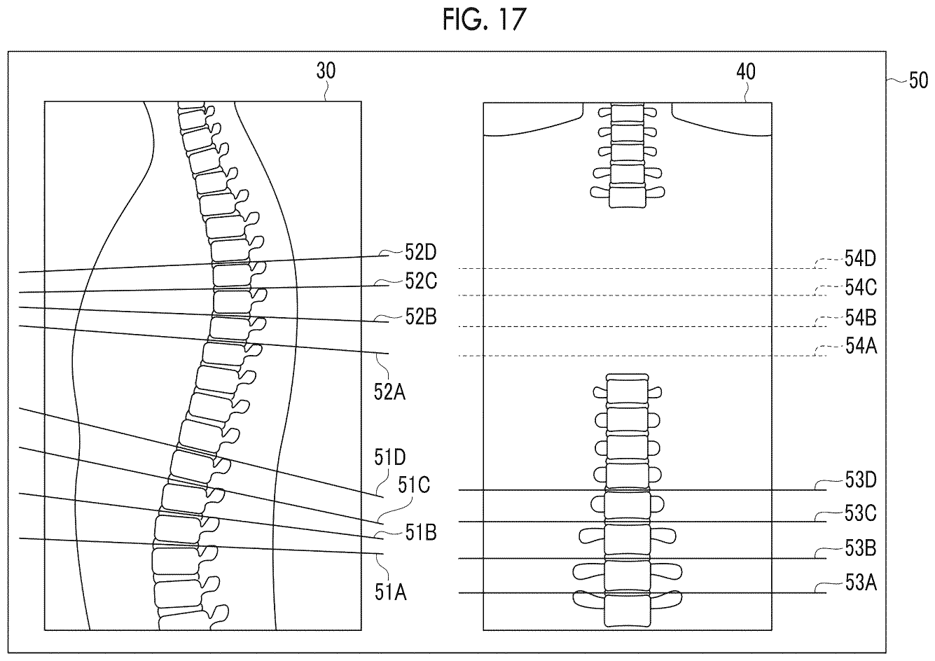

The display control unit 25 displays the scout image including the imaging range set by the imaging range setting unit 24. FIG. 17 is a diagram showing the displayed scout image. As shown in FIG. 17, the sagittal image 30 and the coronal image 40 are displayed on a display screen 50 for the scout image. In the sagittal image 30, indicators 51A to 51D representing the imaging ranges 36A to 36D and indicators 52A to 52D are displayed by straight lines. In the coronal image 40, indicators 53A to 53D representing the imaging ranges, which correspond to the indicators 51A to 51D representing the imaging ranges that are displayed in the sagittal image 30, are displayed. In addition, in the coronal image 40, indicators 54A to 54D representing the imaging ranges, which correspond to the indicators 52A to 52D representing the imaging ranges that are displayed in the sagittal image 30, are displayed. The indicators 53A to 53D are examples of the second indicator, and the indicators 54A to 54D are examples of the first indicator.

Here, in the display screen shown in FIG. 17, only the indicators indicating some imaging ranges are shown, but in practice, indicators indicating the imaging ranges related to all the vertebrae included in the sagittal image 30 or the coronal image 40 are displayed. Additionally, in a case in which at least one of a plurality of sagittal images 30 or a plurality of coronal images 40 is acquired, the sagittal image or the coronal image to be displayed on the display screen 50 can be switched by an instruction via the input device 15.

The imaging ranges displayed in the coronal image 40 include not only the imaging range derived based on the coronal image 40 but also the imaging range derived based on the sagittal image 30. Therefore, in the coronal image 40, the indicator representing the imaging range derived based on the coronal image 40 and the indicator representing the imaging range derived based on the sagittal image 30 may be displayed in a distinguishable manner. In FIG. 17, the indicators 53A to 53D representing the imaging ranges derived based on the coronal image 40 are indicated by solid lines, and the indicators 54A to 54D representing the imaging ranges derived based on the sagittal image 30 are indicated by dashed lines. Instead of using different line types, different colors may be used between the indicators 53A to 53D and the indicators 54A to 54D. The indicators 54A to 54D are examples of the first indicator, and the indicators 53A to 53D are examples of the second indicator.

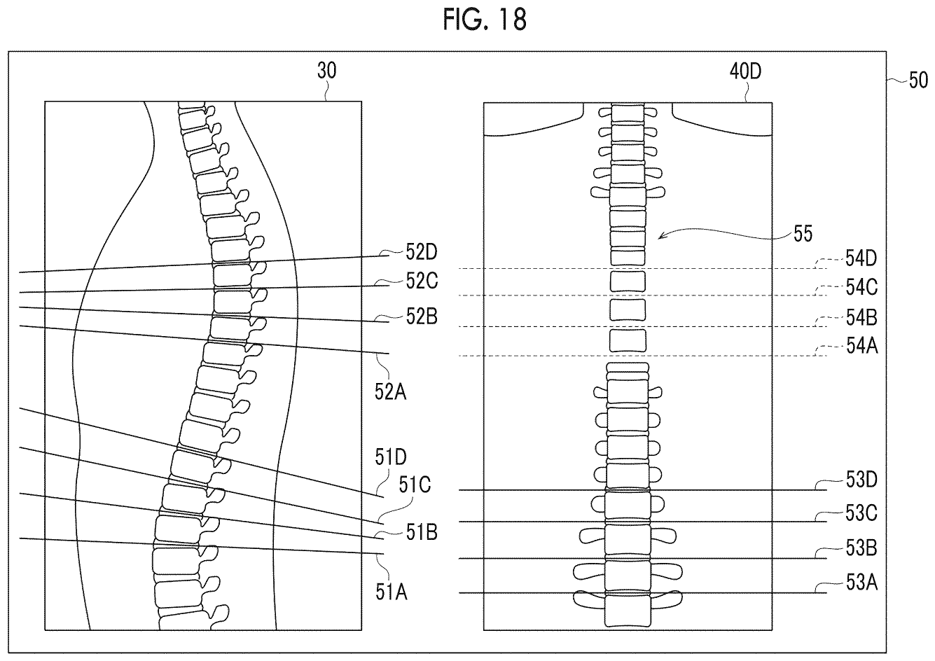

In addition, since some vertebrae are not included in the coronal image as the scout image, it is difficult to understand a relationship between the displayed imaging ranges and the vertebrae. Therefore, as shown in FIG. 18, a virtual coronal image 40D including vertebrae 55 that are not included in the coronal image 40 may be displayed, and indicators representing the imaging ranges may be displayed on the virtual coronal image 40D.

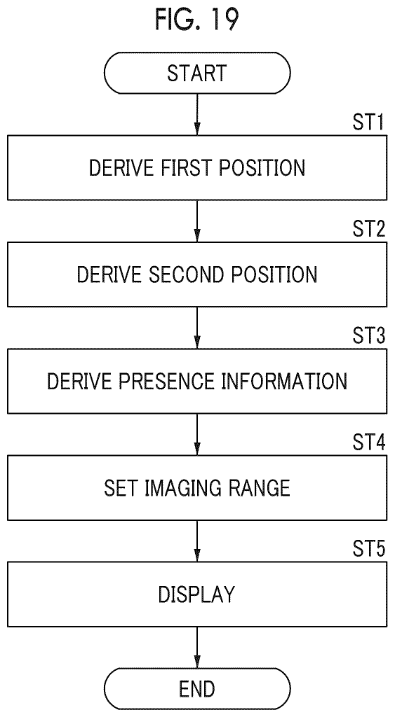

Next, the processing performed in the first embodiment will be described. FIG. 19 is a flowchart showing the processing performed in the first embodiment. It is assumed that the scout images have already been acquired and stored in the storage unit 13.

First, the first derivation unit 21 derives the first position of the structure included in the plurality of first tomographic images respectively representing the plurality of tomographic planes in the first direction for the subject H (step ST1). That is, the landmark of the vertebra included in the sagittal image 30 is derived. Additionally, the second derivation unit 22 derives the second position of the structure included in the second tomographic image representing at least one tomographic plane in the second direction for the subject H (step ST2). That is, the landmark of the vertebra included in the coronal image 40 is derived. The processing of steps ST1 and ST2 may be performed in parallel, or the processing of step ST2 may be performed prior to the processing of step ST1.

Next, the third derivation unit 23 derives the presence information related to the structure in the tomographic plane in the second direction based on the landmark derived from the sagittal image 30 and the landmark derived from the coronal image 40 (step ST3). That is, the inclination and the presence range of the intervertebral disc in the coronal plane are derived as the presence information related to the structure, that is, the intervertebral disc, based on the landmark of the vertebra included in the sagittal image 30 and the landmark of the vertebra included in the coronal image 40.

Subsequently, the imaging range setting unit 24 sets the imaging range for the main imaging based on the presence information related to the intervertebral disc derived by the third derivation unit 23 (step ST4). Further, the display control unit 25 displays at least the coronal image 40 on the display 14 together with the indicator indicating the set imaging range, that is, the straight line (step ST5), and the processing ends. The display control unit 25 may display the coronal image 40 and the sagittal image 30 on the display 14 at the same time together with the straight line or may switch between and display the coronal image 40 and the sagittal image 30 in response to a switching instruction input by the user.

As described above, in the first embodiment, the imaging range is set based on the sagittal image 30 and the coronal image 40. Therefore, even in a case in which some of the vertebrae are not included in the coronal image 40 because of a small number of images captured as in scout imaging, the imaging range for the next imaging in the coronal plane can be set using the sagittal image 30. Accordingly, according to the first embodiment, the imaging range for the MRI image in the main imaging can be accurately set using the scout images.

In addition, in the present embodiment, the presence information related to the structure, that is, the intervertebral disc, is derived based on the landmark of the vertebra included in each of the sagittal image 30 and the coronal image 40, and the imaging range for the next imaging is set based on the presence information. Therefore, the presence information can be accurately derived using the features of the structure which is an imaging target, thereby enabling more accurate setting of the imaging range for the main imaging.

Additionally, in a case in which the indicator (straight line) indicating the imaging range is displayed on the coronal image 40, the indicator representing the imaging range derived based on the coronal image 40 and the indicator representing the imaging range derived based on the sagittal image 30 are displayed in a distinguishable manner. Therefore, the operator who views the coronal image 40 can confirm how the imaging ranges have been set.

In addition, in a case in which the indicator indicating the imaging range is displayed on the coronal image 40, the indicator is displayed on the virtual coronal image 40D including a virtual vertebra for the structure not included in the coronal image 40, thereby making it easier to understand the positional relationship between the vertebra and the imaging range.

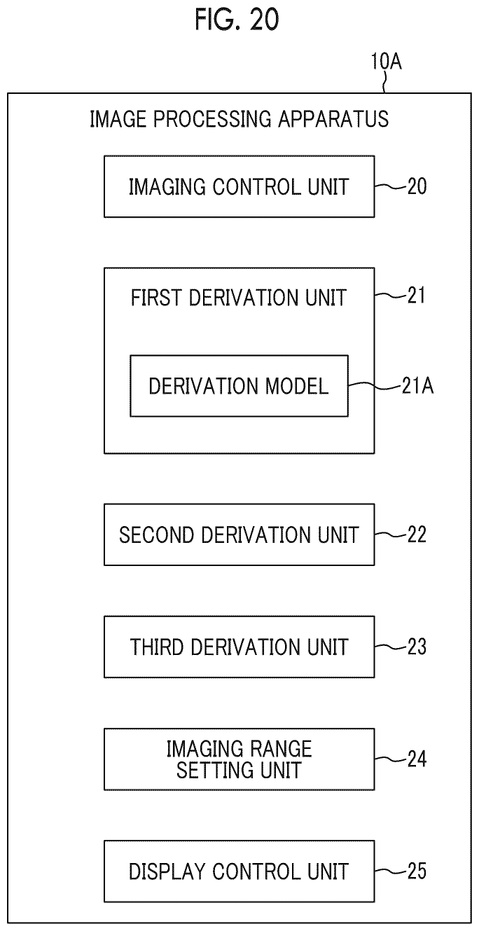

Next, a second embodiment of the present disclosure will be described. FIG. 20 is a functional configuration diagram of an image processing apparatus according to the second embodiment. In the second embodiment, the same components as those in the first embodiment are assigned the same reference numerals, and a detailed description thereof will not be repeated here. An image processing apparatus 10A according to the second embodiment is different from the first embodiment in that a pseudo three-dimensional image including a plurality of tomographic images with a smaller slice thickness or slice interval than that of the sagittal image is derived from the plurality of sagittal images, and the imaging range is set using the pseudo three-dimensional image. Therefore, in the second embodiment, the first derivation unit 21 comprises a derivation model 21A.

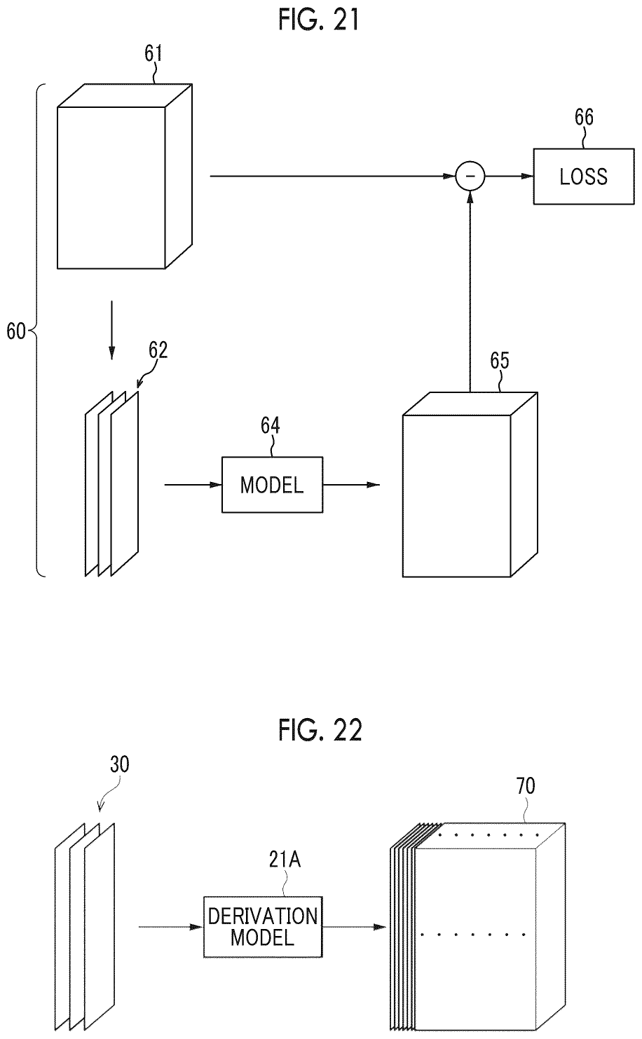

The derivation model 21A is a machine learning model that derives the pseudo three-dimensional image from the plurality of first tomographic images, that is, the plurality of sagittal images. FIG. 21 is a diagram showing training data used for machine learning of the derivation model 21A. As shown in FIG. 21, as training data 60, an MRI image 61 with a small slice thickness or slice interval, which has been acquired through the actual main imaging, and several (here, three) sagittal images 62 obtained by thinning out slices from the MRI image 61 are used. The MRI image 61 consists of a plurality of sagittal images with a smaller slice interval than that of the scout image in a direction perpendicular to the sagittal plane. The direction perpendicular to the sagittal plane refers to the imaging direction of the sagittal image. For example, the direction perpendicular to the sagittal plane indicates a direction of an intersection line between the sagittal plane and the axial plane or a left-right direction of the subject. The number of sagittal images 62 after thinning out the slices from the MRI image 61 need only be the same as the number of sagittal images 30 acquired through the scout imaging. For example, in a case in which three sagittal images are acquired as the scout images, the slices need only be thinned out such that three sagittal images 62 remain from the MRI image 61.

Upon the machine learning of the derivation model 21A, the sagittal images 62 are input to a model 64, and a pseudo three-dimensional image 65 is output from the model 64. Then, a difference between the pseudo three-dimensional image 65 and the MRI image 61 is derived as a loss 66, and learning is repeatedly performed until a predetermined condition is satisfied, thereby constructing the derivation model 21A. The predetermined condition is set to be either until the loss 66 reaches or falls below a threshold value or until a predetermined number of learning operations ends, but the present disclosure is not limited thereto.

As a result, in a case in which the sagittal images 30 acquired as the scout images are input to the derivation model 21A, the pseudo three-dimensional image with a denser slice interval in the direction perpendicular to the sagittal plane than that of the scout image is output.

FIG. 22 is a diagram illustrating the processing performed by the first derivation unit 21 in the second embodiment. In the second embodiment, the first derivation unit 21 uses the derivation model 21A to derive a pseudo three-dimensional image 70 from several (here, three) sagittal images 30 acquired as the scout images. The pseudo three-dimensional image 70 consists of more sagittal images with a smaller slice interval than that of the sagittal image 30 acquired as the scout image.

Therefore, by using the pseudo three-dimensional image 70, the landmarks derived for the same vertebra are positioned closer to each other in the direction perpendicular to the sagittal plane than those in the scout images are. Accordingly, by using the pseudo three-dimensional image 70, it is possible to more accurately derive the presence information, that is, the direction and the presence range of the intervertebral disc or the vertebra, as compared to a case in which the sagittal image 30 acquired as the scout image is used. As a result, in the second embodiment, the imaging range setting unit 24 can more accurately set the imaging range for the main imaging.

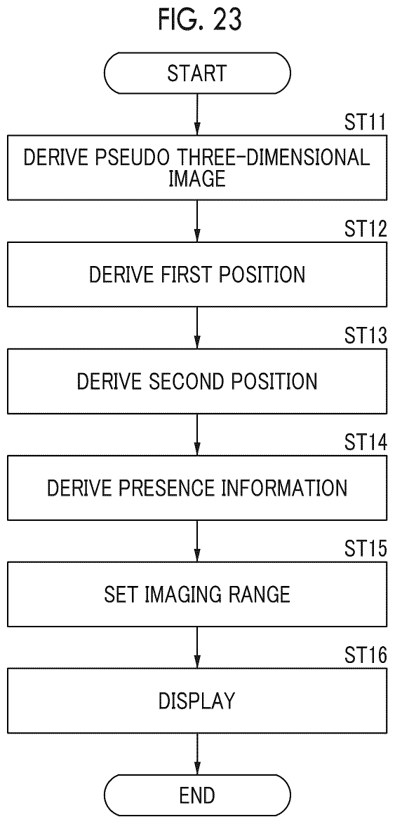

Next, the processing performed in the second embodiment will be described. FIG. 23 is a flowchart showing the processing performed in the second embodiment. It is assumed that the scout images have already been acquired and stored in the storage unit 13.

First, the first derivation unit 21 uses the derivation model 21A to derive the pseudo three-dimensional image of the subject from the plurality of first tomographic images (step ST11). That is, the pseudo three-dimensional image 70 is derived from several sagittal images 30. Then, the first derivation unit 21 derives the first position of the structure in the pseudo three-dimensional image 70 (step ST12). That is, the landmark of the vertebra in the sagittal image included in the pseudo three-dimensional image 70 is derived.

Next, the second derivation unit 22 derives the second position of the structure included in the coronal image 40 (step ST13). That is, the landmark of the vertebra included in the coronal image 40 is derived. The processing of steps ST11 and ST12 and the processing of step ST13 may be performed in parallel, or the processing of step ST13 may be performed prior to the processing of steps ST11 and ST12.

Next, the third derivation unit 23 derives the presence information related to the structure in the tomographic plane in the second direction based on the landmark derived from the pseudo three-dimensional image 70 and the landmark derived from the coronal image 40 (step ST14). That is, the inclination and the presence range of the intervertebral disc or the vertebra in the coronal plane are derived as the presence information based on the landmark of the vertebra included in the sagittal image in the pseudo three-dimensional image 70 and the landmark of the vertebra included in the coronal image 40.

Subsequently, the imaging range setting unit 24 sets the imaging range for the main imaging based on the presence range related to the intervertebral disc or the vertebra derived by the third derivation unit 23 (step ST15). Further, the display control unit 25 displays at least the coronal image 40 on the display 14 together with the indicator indicating the set imaging range, that is, the straight line (step ST16), and the processing ends.

In the above-described first and second embodiments, the intervertebral disc or the vertebra is used as the structure inside the subject, but the present disclosure is not limited thereto. For example, the structure inside the subject can also be set to kidneys. Hereinafter, processing in a case in which the structure inside the subject is the kidneys will be described as a third embodiment.

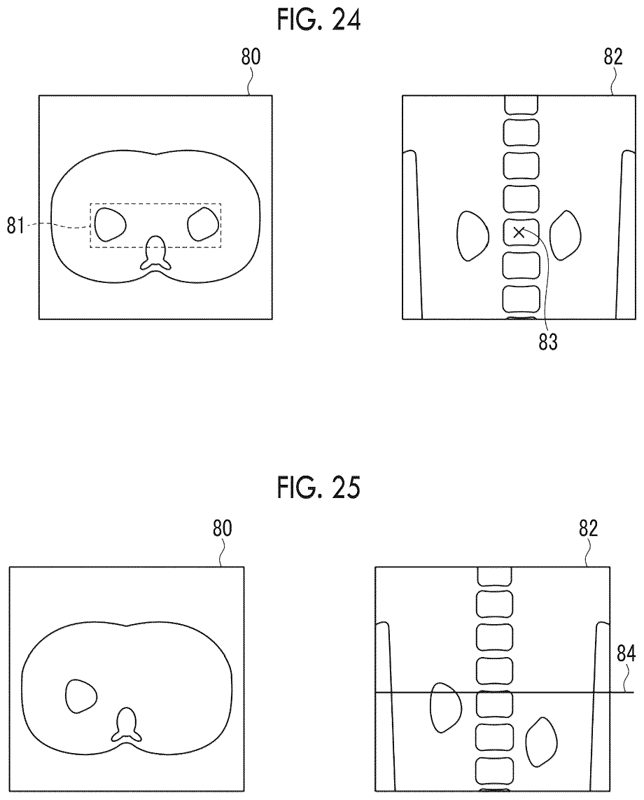

FIG. 24 is a diagram illustrating the setting of the imaging range using the scout image during MRI imaging of the kidneys. As shown in FIG. 24, in a case of capturing an MRI image of the kidneys, an axial image 80 in the axial plane and a coronal image 82 in the coronal plane are acquired as scout images, and a kidney range 81 in a direction perpendicular to the coronal plane is set as the imaging range from the axial image 80, and an imaging center position 83 is set as the imaging range from the coronal image 82. The direction perpendicular to the coronal plane indicates the anterior-posterior direction of the subject.

Meanwhile, in a case in which two kidneys have different positions in a direction perpendicular to the axial plane or one kidney has a size smaller than that of the other kidney, the two kidneys may not be included in the axial image 80. For example, as shown in FIG. 25, in a case in which the left and right kidneys have different positions in the direction perpendicular to the axial plane, only the right kidney is included while the left kidney is not included in the axial image 80, assuming that the axial plane of the axial image 80 corresponds to a slice plane 84 indicated by a straight line in the coronal image 82. In such a case, it is not possible to derive the presence range of the axial plane of the kidneys for the main imaging based only on the axial image 80.

In a case in which the kidneys are set as the imaging target, the positioning is performed such that the kidneys are included in the coronal plane during the scout imaging for acquiring the scout image. Therefore, the coronal image as the scout image includes two kidneys. In the third embodiment, the presence range of the coronal plane of the two kidneys is derived from a plurality of coronal images, and the imaging range for the main imaging is set based on the presence range of the coronal plane.

It should be noted that the functional configuration of the image processing apparatus in the third embodiment is the same as the functional configuration of the image processing apparatus described in the above first embodiment, and only the processing performed by each unit is different. Therefore, a detailed description of the functional configuration will not be repeated here.

In the third embodiment, the first derivation unit 21 derives the first position of the structure included in the plurality of first tomographic images respectively representing the plurality of tomographic planes in the first direction for the subject H. In the third embodiment, the first direction is a direction perpendicular to the coronal plane, the plurality of tomographic planes in the first direction are coronal planes, and the first tomographic image is a coronal image. In addition, the first derivation unit 21 derives kidney regions included in the plurality of coronal images. The kidney region derived from the coronal image is an example of the first position. Therefore, in the third embodiment, the first derivation unit 21 uses a detection model that has undergone machine learning to detect the kidney region from the MRI image.

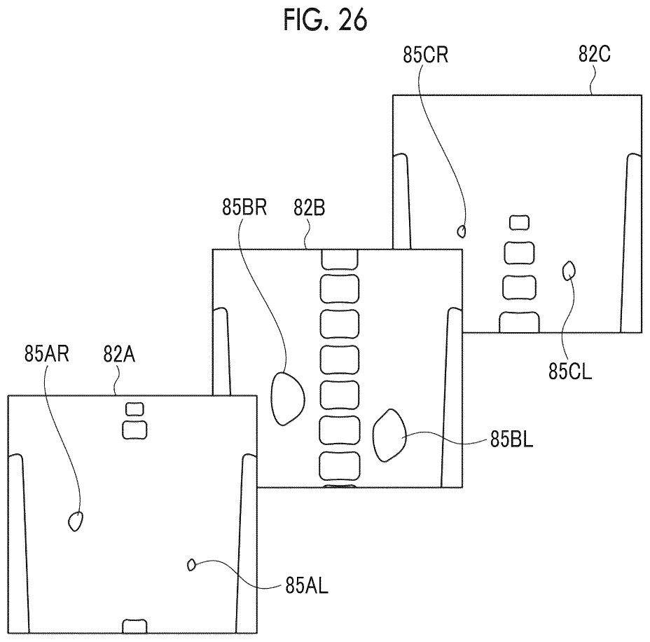

FIG. 26 is a diagram illustrating the derivation of the kidney region from the coronal image in the third embodiment. In the third embodiment, it is assumed that three coronal images 82A to 82C are acquired. The coronal images 82A to 82C show tomographic images of the coronal plane from an anterior side to a posterior side of the human body in this order. As shown in FIG. 26, the first derivation unit 21 derives left and right kidney regions 85AL and 85AR from the coronal image 82A, derives left and right kidney regions 85BL and 85BR from the coronal image 82B, and derives left and right kidney regions 85CL and 85CR from the coronal image 82C. Since the kidney has a shape like a broad bean, as shown in FIG. 26, the kidney regions 85AL, 85AR, 85CL, and 85CR detected in the coronal images 82A and 82C on the anterior side and the posterior side of the human body are smaller in size than the kidney regions 85BL and 85BR detected in the coronal image 82B.

In the third embodiment, the second derivation unit 22 derives the second position of the structure included in the second tomographic image representing at least one tomographic plane in the second direction for the subject H. In the third embodiment, the second direction is a direction perpendicular to the axial plane, the at least one tomographic plane in the second direction is an axial plane, and the second tomographic image is an axial image. Additionally, the second derivation unit 22 derives a kidney region included in at least one axial image. The kidney region derived from the axial image is an example of the second position. Therefore, in the third embodiment, the second derivation unit 22 uses a detection model that has undergone machine learning to detect the kidney region from the MRI image.

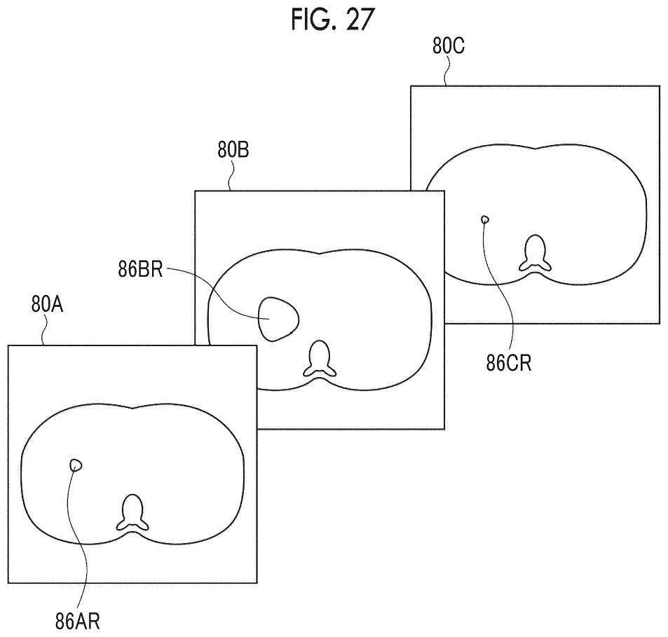

FIG. 27 is a diagram illustrating the derivation of the kidney region from the axial image in the third embodiment. In the third embodiment, it is assumed that three axial images 80A to 80C are acquired. The axial images 80A to 80C show tomographic images of the axial plane from an upper side to a lower side of the human body in this order. The left kidney of the human body is not included in the axial images 80A to 80C shown in FIG. 27. As shown in FIG. 27, the second derivation unit 22 derives a right kidney region 86AR from the axial image 80A, derives a right kidney region 86BR from the axial image 80B, and derives a right kidney region 86CR from the axial image 80C. As shown in FIG. 27, only the kidney region 86AR in an upper portion of the right kidney is detected from the axial image 80A. Only the kidney region 86BR near the center of the right kidney is detected from the axial image 80B. Only the kidney region 86CR in a lower portion of the right kidney is detected from the axial image 80C.

In the third embodiment, the third derivation unit 23 derives the presence information related to the kidneys in the direction perpendicular to the coronal plane based on the kidney region included in the coronal image derived by the first derivation unit 21 and the kidney region included in the axial image derived by the second derivation unit 22. First, the derivation of the presence information related to the kidney included in the coronal image will be described. The direction perpendicular to the coronal plane indicates the anterior-posterior direction of the human body.

First, the third derivation unit 23 derives the presence information related to the kidneys in the coronal plane using the kidney region derived in the coronal image. That is, the position and the presence range of the kidneys are derived as the presence information based on the sizes of the kidney regions 85AL, 85AR, 85BL, 85BR, 85CL, and 85CR respectively derived in the coronal images 82A to 82C and the slice intervals of the coronal images 82A to 82C.

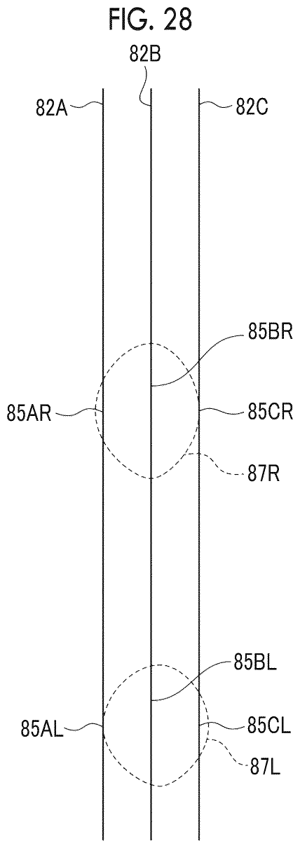

FIG. 28 is a diagram illustrating the derivation of the presence information related to the kidneys included in the coronal image. As shown in FIG. 28, the third derivation unit 23 derives the position and the presence range of the left kidney as presence information 87L based on the sizes of the kidney regions 85AL, 85BL, and 85CL respectively derived in the coronal images 82A to 82C and the slice intervals of the coronal images 82A to 82C. In addition, the third derivation unit 23 derives the position and the presence range of the right kidney as presence information 87R based on the sizes of the kidney regions 85AR, 85BR, and 85CR respectively derived in the coronal images 82A to 82C and the slice intervals of the coronal images 82A to 82C.

In order to derive the presence information, the third derivation unit 23 need only use a derivation model that has undergone machine learning to derive the presence range related to the kidneys based on, for example, the slice interval of the coronal images and the size of the kidney region included in each coronal image, but the present disclosure is not limited thereto.

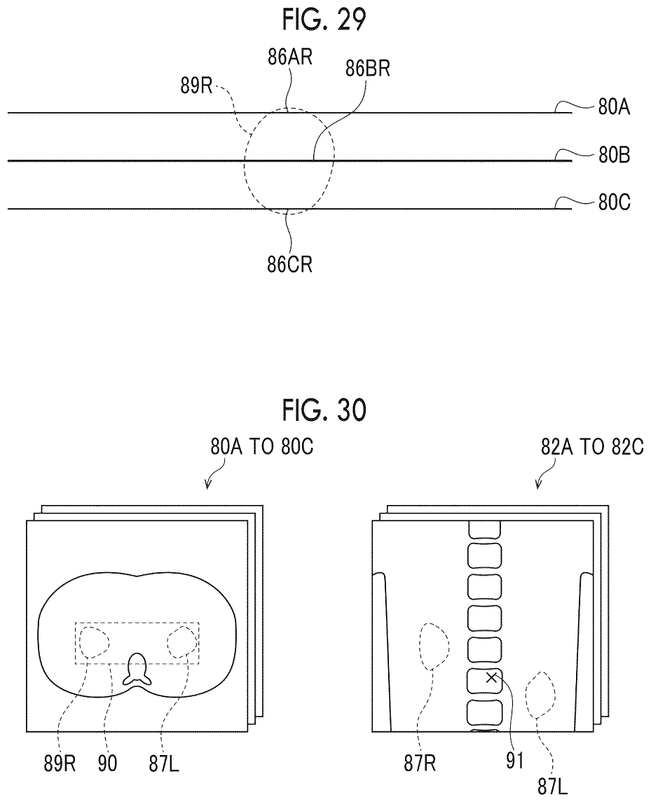

Additionally, the third derivation unit 23 derives the presence information related to the kidney in the axial plane by using the kidney region derived in the axial image. FIG. 29 is a diagram illustrating the derivation of the presence information related to the kidney included in the axial image. As shown in FIG. 29, the third derivation unit 23 derives the position and the presence range of the kidney as the presence information based on the sizes of kidney regions 86AR, 86BR, and 86CR respectively derived in the axial images 80A to 80C and the slice intervals of the axial images 80A to 80C.

In order to derive the presence information, the third derivation unit 23 need only use a derivation model that has undergone machine learning to derive the presence range of the kidney based on, for example, the slice interval of the axial images and the size of the kidney region included in each axial image, but the present disclosure is not limited thereto.

Here, as shown in FIG. 27, the left kidney is not included in the axial images 80A to 80C. Therefore, the third derivation unit 23 derives presence information 89R only for the right kidney regions included in the axial images 80A to 80C. The third derivation unit 23 derives the presence information related to the left kidney region that cannot be derived from the axial images 80A to 80C by using the presence information 87L related to the left kidney derived from the coronal images 82A to 82C. Specifically, the presence information related to the three-dimensional kidney is derived based on a positional relationship between the kidney region in each of the plurality of coronal images 82A to 82C and the direction perpendicular to the coronal plane of each of the tomographic images, and the presence information corresponding to the axial image in the three-dimensional presence information is corrected using the presence information related to the kidney derived from the axial image.

The correction of the three-dimensional presence information may be performed simply by replacing the three-dimensional presence information with the presence information derived from the axial image, but the correction may also be performed such that the correction propagates from the replaced region by using a derivation model, a specific calculation expression, or the like. The correction may be performed such that, for example, in a case in which the direction perpendicular to the coronal image is a Y-axis, the Y coordinates of a presence region derived from the coronal image and a presence region derived from the corresponding axial image may be replaced, and the Y coordinate of a presence region derived from the adjacent coronal image is corrected based on the amount of change in the replaced Y coordinate. As a result, it is possible to derive a more appropriate presence range even in a region where the axial image has not been acquired.

The imaging range setting unit 24 sets the imaging range for the main imaging based on the presence information related to the kidneys derived by the third derivation unit 23. Specifically, since the acquired coronal image 82 and axial image 80 are scout images, the imaging center position for the kidneys and the kidney range in the anterior-posterior direction in a case of performing the main imaging of the subject H are set as the imaging ranges.

FIG. 30 is a diagram illustrating the setting of the imaging range in the third embodiment. As shown in FIG. 30, since only the right kidney is included in the axial images 80A to 80C, the imaging range setting unit 24 sets a kidney range 90 in the anterior-posterior direction of the human body by using not only the presence information 89R related to the right kidney but also the presence information 87L related to the left kidney derived from the coronal images 82A to 82C. In addition, as shown in FIG. 30, an imaging center position 91 is derived as the imaging range based on the presence information 87R related to the right kidney and the presence information 87L related to the left kidney, which are derived in the coronal images 82A to 82C.

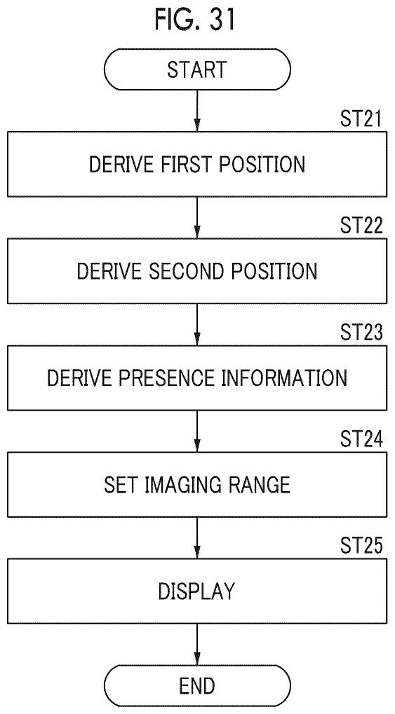

Next, the processing performed in the third embodiment will be described. FIG. 31 is a flowchart showing the processing performed in the third embodiment. It is assumed that the scout images have already been acquired and stored in the storage unit 13.

First, the first derivation unit 21 derives the first position of the structure included in the plurality of first tomographic images respectively representing the plurality of tomographic planes in the first direction for the subject H (step ST21). That is, the kidney regions included in each of the coronal images 82A to 82C are derived. Additionally, the second derivation unit 22 derives the second position of the structure included in at least one second tomographic image representing at least one tomographic plane in the second direction for the subject H (step ST22). That is, the kidney region included in each of the axial images 80A to 80C is derived. The processing of steps ST21 and ST22 may be performed in parallel, or the processing of step ST22 may be performed prior to the processing of step ST21.

Next, the third derivation unit 23 derives the presence information related to the structure in the tomographic plane in the second direction based on the kidney region derived from the coronal image and the kidney region derived from the axial image (Step ST23). That is, the presence information related to the kidneys in the axial plane is derived based on the kidney regions included in the coronal images 82A to 82C and the kidney regions included in the axial images 80A to 80C.

Subsequently, the imaging range setting unit 24 sets the imaging range for the next imaging based on the presence information related to the kidneys derived by the third derivation unit 23 (step ST24). Further, the display control unit 25 assigns indicators indicating the set imaging ranges, that is, the kidney range 90 in the anterior-posterior direction of the human body and the imaging center position 91 in the coronal cross section, to each of the axial images 80A to 80C and the coronal images 82A to 82C and displays the indicators on the display 14 (step ST25), and the processing ends.

In each of the above-described embodiments, the image processing apparatus according to the present disclosure is applied to the MRI apparatus, but the present disclosure is not limited thereto. The image processing apparatus according to the present disclosure may be applied to an imaging apparatus that acquires a scout image for setting an imaging range before the main imaging, such as a CT apparatus.

In addition, in each of the above-described embodiments, the image processing apparatus comprises the imaging control unit 20, but the present disclosure is not limited thereto. The imaging control unit 20 may be provided separately from the image processing apparatus.

Additionally, in the above-described first and second embodiments, a plurality of coronal images are acquired as scout images, and in the above-described third embodiment, a plurality of axial images are acquired as scout images, but the present disclosure is not limited thereto. In the above-described first and second embodiments, one coronal image may be acquired as the scout image. Further, in the third embodiment, one axial image may be acquired as the scout image.

Moreover, in each of the above-described embodiments, the console 4 incorporates the image processing apparatus according to the present embodiment, but the present disclosure is not limited thereto. The image processing apparatus according to the present embodiment may be an apparatus connected to the console 4 via a network. That is, the image processing apparatus need not be connected to the gantry 2 and the patient table 3 and need not control the gantry 2 and the patient table 3.

In this embodiment, each process is executed on an arbitrary computer. The arbitrary computer may execute these processes by means of a processor as hardware, a program as software, or a combination of the processor and the program. In such a case, the processor is configured to execute the various processes in this embodiment in cooperation with the program and may function as each unit or means in this embodiment. In addition, the order in which the processor executes these processes is not limited to the order described in this embodiment and may be changed as appropriate. The arbitrary computer may be a general-purpose computer, a computer for a specific purpose, a workstation, or any other system capable of executing each process.

The processor may be configured by one or more hardware, and the type of hardware is not limited. For example, the processor may comprise at least one of programmable logic devices such as CPUs (Central Processing Units), MPUs (Micro Processing Units), and FPGAs (Field Programmable Gate Arrays); dedicated circuits for performing specific processes such as ASICs (Application Specific Integrated Circuits); and other hardware such as a GPU (Graphics Processing Unit) and an NPU (Neural Processing Unit). The hardware may also be a combination of different types of hardware. When multiple hardware are configured to execute one or more processes of a processor, the said multiple hardware may exist in devices that are physically separate from each other, or in the same device. In any embodiment, the order of each process by the processor is not limited to the order described above and may be changed as appropriate. The hardware is configured by an electric circuit (circuitry) etc. that combines circuit elements such as semiconductor devices.

Furthermore, the program may be firmware or software such as microcode. The program may also be a group of program modules, each function of which may be performed by a processor configured to execute each of the program modules. The program may be program code or code segments stored on one or more non-transitory computer-readable media (e.g., storage media or other storage). The program may be stored in separate non-transitory computer-readable media located on devices that are physically separate from each other. The program code or code segments may represent any combination of procedures, functions, subprograms, routines, subroutines, modules, software packages, classes, instructions, data structures, or program statements. The program code or code segments may be connected to other code segments or hardware circuits by sending or receiving information, data, arguments, parameters, or memory contents.

In the above embodiment, it is explained that the image processing program 12 is stored (installed) in advance in the storage unit 13, but this is not limited to this. The image processing program 12 may be provided in a form recorded on a recording medium such as a CD-ROM (Compact Disc Read Only Memory), DVD-ROM (Digital Versatile Disc Read Only Memory), and USB (Universal Serial Bus) memory. In addition, the image processing program 12 may be provided in a form that the image processing program 12 is downloaded from an external device via a network.

The technology of this disclosure also extends to all types of program products. Program products include all types of products for providing programs. For example, program products include programs provided via networks such as the Internet, and non-temporary computer readable storage media such as CD-ROMs, DVDs, and USB memory devices that store programs.

The supplementary claims of the present disclosure will be described below.

### Supplementary Claim 1

An image processing apparatus comprising:
a processor,
in which the processor is configured to set an imaging range based on a plurality of first tomographic images respectively representing a plurality of tomographic planes in a first direction for a subject and at least one second tomographic image representing a tomographic plane in a second direction intersecting the first direction.

### Supplementary Claim 2

The image processing apparatus according to Supplementary Claim 1,
in which the processor is configured to:
   derive presence information related to a structure inside the subject in the tomographic plane in the second direction based on the first tomographic image and the second tomographic image; and
   set the imaging range based on the presence information.

### Supplementary Claim 3

The image processing apparatus according to Supplementary Claim 2,
in which, in a case in which a plurality of the structures are included in the subject,
the processor is configured to:
   derive the presence information for a structure included in the second tomographic image based on the second tomographic image;
   set an imaging range for the structure included in the second tomographic image, based on the presence information derived based on the second tomographic image;
   derive the presence information for a structure not included in the second tomographic image based on the first tomographic image; and
   set an imaging range for the structure not included in the second tomographic image, based on the presence information derived based on the first tomographic image.

### Supplementary Claim 4

The image processing apparatus according to Supplementary Claim 2 or 3,
in which the processor is configured to:
   derive a first position representing a position of the structure included in the plurality of first tomographic images;
   derive a second position representing a position of the structure included in the second tomographic image; and
   derive the presence information based on the first position and the second position.

### Supplementary Claim 5

The image processing apparatus according to any one of Supplementary Claims 1 to 4,
in which the processor is configured to:
   display the second tomographic image on a display; and
   display an indicator representing the imaging range in the second tomographic image.

### Supplementary Claim 6

The image processing apparatus according to Supplementary Claim 5,
in which the processor is configured to display a first indicator representing the imaging range derived based on the first tomographic image and a second indicator representing the imaging range derived based on the second tomographic image in a distinguishable manner.

### Supplementary Claim 7

The image processing apparatus according to Supplementary Claim 6,
in which the processor is configured to:
   in a case in which the imaging range derived based on the first tomographic image is outside a range of the second tomographic image, display a virtual second tomographic image including a virtual structure in the second direction on the display; and
display the first indicator and the second indicator on the virtual second tomographic image.

### Supplementary Claim 8

The image processing apparatus according to any one of Supplementary Claims 1 to 7,
in which the processor is configured to:
   derive, from N first tomographic images, a pseudo three-dimensional image of the subject including M tomographic images with a smaller slice thickness than that of the first tomographic image, where M>N; and
   derive the imaging range based on the pseudo three-dimensional image.

### Supplementary Claim 9

The image processing apparatus according to any one of Supplementary Claims 1 to 8,
in which the subject includes a structure, and the structure is a vertebra or an intervertebral disc of the subject, and
the imaging range is an imaging plane of at least one of the vertebra or the intervertebral disc.

### Supplementary Claim 10

The image processing apparatus according to Supplementary Claim 9,
in which the tomographic plane in the first direction is a sagittal plane, and
the tomographic plane in the second direction is a coronal plane.

### Supplementary Claim 11

The image processing apparatus according to Supplementary Claim 10,
in which the processor is configured to set the imaging plane such that a difference in inclination between imaging planes of adjacent vertebrae or intervertebral discs falls within a predetermined range.

### Supplementary Claim 12

The image processing apparatus according to Supplementary Claim 11,
in which the processor is configured to, in a case in which the difference in inclination between the imaging planes of the adjacent vertebrae or intervertebral disc exceeds the predetermined range, correct the inclination of the imaging plane such that the difference in inclination between the imaging planes of the adjacent vertebrae or intervertebral disc falls within the predetermined range.

### Supplementary Claim 13

The image processing apparatus according to Supplementary Claim 11,
in which the processor is configured to:
   define centerlines of a plurality of the vertebrae or the intervertebral discs; and
   set the imaging plane to be orthogonal to the centerline.

### Supplementary Claim 14

The image processing apparatus according to any one of Supplementary Claims 1 to 8,
in which the subject includes a structure, and the structure is kidneys.

### Supplementary Claim 15

The image processing apparatus according to Supplementary Claim 14,
in which the tomographic plane in the first direction is a coronal plane,
the tomographic plane in the second direction is an axial plane, and
the imaging range is a range in which the kidneys are present.

### Supplementary Claim 16

An image processing method comprising:
causing a computer to execute:
   setting an imaging range based on a plurality of first tomographic images respectively representing a plurality of tomographic planes in a first direction for a subject and at least one second tomographic image representing a tomographic plane in a second direction intersecting the first direction.

### Supplementary Claim 17

An image processing program for causing a computer to execute a procedure comprising:
setting an imaging range based on a plurality of first tomographic images respectively representing a plurality of tomographic planes in a first direction for a subject and at least one second tomographic image representing a tomographic plane in a second direction intersecting the first direction.

What is claimed is:

1. An image processing apparatus comprising:
a processor,
wherein the processor is configured to set an imaging range based on a plurality of first tomographic images respectively representing a plurality of tomographic planes in a first direction for a subject and at least one second tomographic image representing a tomographic plane in a second direction intersecting the first direction.

2. The image processing apparatus according to claim 1,
wherein the processor is configured to:
derive presence information related to a structure inside the subject in the tomographic plane in the second direction based on the first tomographic image and the second tomographic image; and
set the imaging range based on the presence information.

3. The image processing apparatus according to claim 2,
wherein, in a case in which a plurality of the structures are included in the subject,
the processor is configured to:
derive the presence information for a structure included in the second tomographic image based on the second tomographic image;
set an imaging range for the structure included in the second tomographic image, based on the presence information derived based on the second tomographic image;
derive the presence information for a structure not included in the second tomographic image based on the first tomographic image; and
set an imaging range for the structure not included in the second tomographic image, based on the presence information derived based on the first tomographic image.

4. The image processing apparatus according to claim 2,
wherein the processor is configured to:
derive a first position representing a position of the structure included in the plurality of first tomographic images;
derive a second position representing a position of the structure included in the second tomographic image; and
derive the presence information based on the first position and the second position.

5. The image processing apparatus according to claim 1,
wherein the processor is configured to:
display the second tomographic image on a display; and
display an indicator representing the imaging range in the second tomographic image.

6. The image processing apparatus according to claim 5,
wherein the processor is configured to display a first indicator representing the imaging range derived based on the first tomographic image and a second indicator representing the imaging range derived based on the second tomographic image in a distinguishable manner.

7. The image processing apparatus according to claim 6,
wherein the processor is configured to:
in a case in which the imaging range derived based on the first tomographic image is outside a range of the second tomographic image, display a virtual second tomographic image including a virtual structure in the second direction on the display; and
display the first indicator and the second indicator on the virtual second tomographic image.

8. The image processing apparatus according to claim 1,
wherein the processor is configured to:
derive, from N first tomographic images, a pseudo three-dimensional image of the subject including M tomographic images with a smaller slice thickness than that of the first tomographic image, where M>N; and
derive the imaging range based on the pseudo three-dimensional image.

9. The image processing apparatus according to claim 1,
wherein the subject includes a structure, and the structure is a vertebra or an intervertebral disc of the subject, and
the imaging range is an imaging plane of at least one of the vertebra or the intervertebral disc.

10. The image processing apparatus according to claim 9,
wherein the tomographic plane in the first direction is a sagittal plane, and
the tomographic plane in the second direction is a coronal plane.

11. The image processing apparatus according to claim 10,
wherein the processor is configured to set the imaging plane such that a difference in inclination between imaging planes of adjacent vertebrae or intervertebral discs falls within a predetermined range.

12. The image processing apparatus according to claim 11,
wherein the processor is configured to, in a case in which the difference in inclination between the imaging planes of the adjacent vertebrae or intervertebral disc exceeds the predetermined range, correct the inclination of the imaging plane such that the difference in inclination between the imaging planes of the adjacent vertebrae or intervertebral disc falls within the predetermined range.

13. The image processing apparatus according to claim 11,
wherein the processor is configured to:
define centerlines of a plurality of the vertebrae or the intervertebral discs; and
set the imaging plane to be orthogonal to the centerline.

14. The image processing apparatus according to claim 1,
wherein the subject includes a structure, and the structure is kidneys.

15. The image processing apparatus according to claim 14,
wherein the tomographic plane in the first direction is a coronal plane,
the tomographic plane in the second direction is an axial plane, and
the imaging range is a range in which the kidneys are present.

16. An image processing method comprising:
causing a computer to execute:
setting an imaging range based on a plurality of first tomographic images respectively representing a plurality of tomographic planes in a first direction for a subject and at least one second tomographic image representing a tomographic plane in a second direction intersecting the first direction.

17. A non-transitory computer-readable storage medium that stores an image processing program for causing a computer to execute a procedure comprising:
setting an imaging range based on a plurality of first tomographic images respectively representing a plurality of tomographic planes in a first direction for a subject and at least one second tomographic image representing a tomographic plane in a second direction intersecting the first direction.
